(19) United States
(12) Patent Application Publication
(10) Pub. No.: US 2024/0145066 A1
(43) Pub. Date: May 2, 2024

(54) Pervasive and Ubiquitous Decentralized Biocybernetic Adaptation and Biofeedback Training System

(71) Applicant: UNITED STATES OF AMERICA AS REPRESENTED BY THE ADMINISTRATOR OF NASA, WASHINGTON, DC (US)

(72) Inventors: JOHN EDISON MUNOZ CARDONA, Waterloo (CA); CHAD L. STEPHENS, POQUOSON, VA (US); OLAFUR S. PALSSON, CHAPEL HILL, NC (US); ALAN T. POPE, POQUOSON, VA (US)

(21) Appl. No.: 18/515,868

(22) Filed: Nov. 21, 2023

Related U.S. Application Data
(60) Provisional application No. 63/437,232, filed on Jan. 5, 2023.
(60) Provisional application No. 63/420,914, filed on Oct. 31, 2022.

(51) Int. Cl. G16H 20/70; G16H 40/67

(52) U.S. Cl. CPC G16H 20/70; G16H 40/67

(57) ABSTRACT

Aspects relate to systems and methods for providing information about a person's physiological signals to that person (and/or others) as they travel through various venues of their daily work and play life. Ubiquitous physiological feedback incentivizes the person to regulate their cognitive and/or emotional states to better perform various tasks. The feedback also provides them with practice opportunities throughout their day to further develop a psychophysiological self-regulation skill set. Obtaining pervasive physiological feedback throughout the environment may be achieved through the person's use and/or interaction with wearable or otherwise mobile physiological monitoring devices or systems and with other systems that may be embedded in environments through which the traveler visits during the course of their day. Such systems may experience functionality changes in response to an individual's physiological signals via biocybernetic adaptation.

### CROSS-REFERENCE TO RELATED PATENT APPLICATION(S)

This patent application claims the benefit of and priority to U.S. Provisional Patent Application No. 63/426,914, filed on Nov. 21, 2022 and U.S. Provisional Pat. App. 63/437,232, filed on Jan. 5, 2023, the contents of which are hereby incorporated by reference in their entirety for any and all non-limiting purposes. This application is also being filed concurrently with U.S. patent application Ser. No. ______, Docket No. LAR-19961-1, the contents of which are hereby incorporated by reference in their entirety for any and all non-limiting purposes.

### STATEMENT REGARDING FEDERALLY SPONSORED RESEARCH OR DEVELOPMENT

The invention described herein was made in part by an employee of the United States Government and may be manufactured and used by and for the Government of the United States for governmental purposes without the payment of any royalties thereon or therefore.

### BACKGROUND

Currently available technologies designed to foster effective cognitive and/or emotional regulation for individuals in the moment and/or to train cognitive and/or emotional regulation over time are limited to particular settings or environments, such as within a therapy clinic. Technology that fosters effective cognitive and/or emotional regulation in the moment and/or trains cognitive and/or emotional regulation over time is needed across and within multiple settings or environments encountered by individuals throughout their daily lives. Existing biocybernetically adaptive technology encounters multiple problems including, for example, limited settings because conventional biocybernetic systems have been designed to be used under specific conditions and limited or narrow settings or environments, so their training process occurs in a non-continuous fashion, thus limiting the applications use, limiting exposure for individuals to these biocybernetic systems, and limiting a person's ability to experience any benefits of self-regulation training.

Further problems of existing biocybernetically adaptive technology includes feedback modalities because most feedback mechanisms previously proposed for self-regulation through adaptive biofeedback training are limited in terms of overall sense stimuli. Instead, successful self-regulation training needs a multi-sense, rich, and consistent feedback. Adaptation and long-term use may be an issue with present technologies because, to get measurable results using biocybernetic systems to promote self-regulation, multiple sessions are required. A common problem here is that use of most of the biocybernetic systems is short-term due to complications in data acquisition (e.g., sensors level of intrusiveness), computational issues (e.g., processing data in real time), and settings for the final application. Therefore, no learning regarding adaptation is made since only a few sessions are conventionally carried out.

### BRIEF SUMMARY OF THE INVENTION

The present invention is a system and method for providing biocybernetic adaptation and biofeedback training for a user as they interact with their environment. Wearable or environment-embedded components may identify and/or track the user's physiological signals, compute estimates of their cognitive and emotional states, and communicate the estimates to components embedded in the environments/settings through which the traveler moves. The estimates may generate signals to modify aspects of the local environments in such a way as to encourage and/or reinforce neurophysiological self-regulation by the user. Visual, haptic, audio, and similar feedback may be generated via virtual reality devices and/or embedded computing devices within the environment local to the user, such as a vehicle interior and/or a game playing environment.

One embodiment of the invention is a biocybernetic adaptation and biofeedback training system including a plurality of electronic devices associated with a user and a computing device. Each electronic device of the plurality of electronic devices generate a signal associated with a physiological response of the user. The computing device may include at least one processor and memory storing instructions that, when executed by the at least one processor, cause the computing device to receive, from at least one electronic device of the plurality of electronic devices, a first signal associated with a first physiological response of the user. The computing device may then determine, based on the first signal, an estimate of a state of the user and cause, based on the estimate of the state of the user, presentation of an electronic representation of a psychophysiological stimuli via a second electronic device embedded in an environment local to the user. In some cases, the state of the user comprises one or both of a cognitive state and an emotional state and the instructions further cause the computing device to send, to the second electronic device embedded in the environment local to the user, a signal associated with the estimate of the state of the user. The plurality of electronic devices associated with the user may include a smart phone, one or more physiological sensor devices, a smart watch, smart glasses and/or the like. At least one physiological sensor device may generate a signal associated with a physiological state experienced by the user. In some cases, the environment local to the user comprises a vehicle interior. In some cases, the environment local to the user comprises a portion of an electronic gaming system. The second electronic device embedded in the environment local to the user may be a virtual reality display, where the instructions further cause the computing device to cause the virtual reality display to display a visual representation of a locality external to the environment local to the user. In some cases, the environment local to the user comprises an interior space of a vehicle and the locality external to the environment local to the user comprises a space external to the vehicle.

Another embodiment of the invention is a method comprising receiving, from at least one electronic device of a plurality of electronic devices associated with a user, a first signal associated with a first physiological response of the user, determining, based on the first signal, an estimate of a state of the user comprising one or both of a cognitive state of the user and an emotional state of the user, and causing, based on the estimate of the state of the user, presentation of an electronic representation of a psychophysiological stimuli via a second electronic device embedded in an environment local to the user. The method may further include sending, to the second electronic device embedded in the environment local to the user, a signal associated with the estimate of the state of the user. In some cases, the plurality of electronic devices associated with the user comprises a smart phone, at least one physiological sensor devices, and/or other such devices. The at least one physiological sensor device may generate a signal associated with a physiological state experienced by the user. In some cases, the second electronic device embedded in an environment local to the user comprises a virtual reality device associated with a gaming system., where the virtual reality device generates one or more of visual feedback, haptic feedback, and audio feedback.

Yet another embodiment of the invention is a computing device comprising at least one processor and memory storing instructions that, when executed by the at least one processor, cause the computing device to receive, from at least one electronic device of a plurality of electronic devices, a first signal associated with a first physiological response of a user, wherein the plurality of electronic devices comprises one or more of a physiological sensor, a smart watch, and a smart phone, determine, based on the first signal, an estimate of a state of the user, and cause, based on the estimate of the state of the user, presentation of an electronic representation of a psychophysiological stimuli via a second electronic device embedded in an environment local to the user. In some cases, the second electronic device embedded in an environment local to the user comprises a virtual reality device associated with a gaming system and the virtual reality device generates one or more of visual feedback, haptic feedback, and audio feedback.

These and other features, advantages, and objects of the present invention will be further understood and appreciated by those skilled in the art by reference to the following specification, claims, and appended drawings.

### BRIEF DESCRIPTION OF THE SEVERAL VIEWS OF THE DRAWINGS

FIG. 1 is a block diagram of an illustrative biocybernetic adaptation and biofeedback training system, according to aspects described herein;

FIG. 2 is an illustrative method for providing biocybernetic adaptation and biofeedback training; and

FIG. 3 is an illustrative operating environment in which various aspects of the disclosure may be implemented in accordance with one or more aspects described herein.

### DETAILED DESCRIPTION

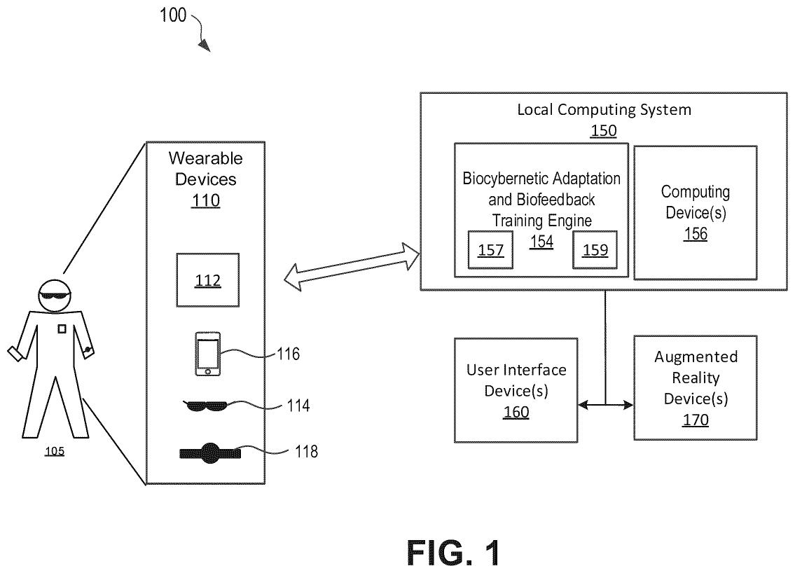

For purposes of description herein, the terms “upper,” “lower,” “right,” “left,” “rear,” “front,” “vertical,” “horizontal,” and derivatives thereof shall relate to the invention as oriented in FIG. 1. However, it is to be understood that the invention may assume various alternative orientations and step sequences, except where expressly specified to the contrary. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

FIG. 1 is a block diagram of an illustrative biocybernetic adaptation and biofeedback training system 100. The biocybernetic adaptation and biofeedback training system 100 may include one or more wearable devices (e.g., one or more physiological sensors 112, a smart phone 116, smart glasses 114, a smart watch 118, etc.) that may be worn, carried, or otherwise utilized by a user 105. The wearable devices 110 may be in wired or wireless communication with a local computing system 150 (e.g., an environmental computing system) that may include a biocybernetic adaptation and biofeedback training engine 154 that may be communicatively be coupled to one or more computing devices 156. The one or more computing devices 156 may include one or more interactive computing devices providing a function, service, and/or other functionality to the user 105. For example, the local computing system may interact with the user 105 via one or more user interface devices 160 (e.g., a user command interface, a display device, gauges, switches, audio output devices, video output devices, lighting sensors, and/or the like). Additionally, the local computing system 150 may also incorporate one or more augmented reality devices, such as virtual reality displays.

The innovation is a method and system that comprises two interacting sets of components: 1) wearable or environment embedded components, such as the wearable devices 110, that track an individual's physiological signals, compute estimates of their cognitive and emotional states and communicate the estimates to 2) other components (e.g., the local computing system 150), in the environments through which the individual 105 moves, that modify aspects of the environments. In some cases, the sensor components may be thought of as a talisman the user 105 carries with them to enable themselves to be reminded of their intentions. And the components in the environment providing the feedback can be thought of as artifacts, which signal to the user 105, the state they intend to achieve. The local system 150 may provide sensory feedback to the individual 105 regarding their states that is designed to encourage them to respond with situationally effective states while in the environments and/or to develop the ability to respond in any future environment with appropriately effective states. The system 100 may be configured by the user 105 to enable achievement of an appropriate psychophysiological persona consistent with the specific context or environment. Illustrative examples, or embodiments, are discussed below in greater detail.

A first embodiment of the innovation is an adaptive virtual ambience technology that comprises a system of wearable components 110 that monitor neural and cardiac signals, where one of the wearable components 110 may compute indices of engagement (e.g., a talisman) and transmits those estimates to a subsystem of the local computing system 150. For example, the local computing system may be integrated into an aircraft flight deck environment (e.g., an artifact). The subsystem may cause a synthetic panorama of the surrounding landscape/skyscape/traffic/terrain to be displayed as a backdrop to flight deck instrumentation on unused interior panel real estate, such as via the augmented reality devices 170, the user interface devices 160, and/or a combination of the user interface devices 160 and the augmented reality devices 170.

Sections of the panorama may be added or removed by the biocybernetic adaptation and biofeedback training engine 154 as requirements for engagement fluctuate, driven by fluctuations in the measured crew engagement state combined with evolving changes in the necessity for engagement in the flight management task. In the extreme case of stripping away the crew's insulation from reality, a crew is virtually suspended at the center of a transparent hemisphere surrounded by the real sights and sounds of flight. A windowless, quiet cockpit represents the other extreme. The current ambience of a commercial flight deck insulates the crew in a technological “cocoon” which can serve to prevent them from experiencing the situation as potentially jeopardous, and thus fosters unwarranted complacency and disengagement. In this illustrative example, the biocybernetic adaptation and biofeedback training engine 154 either peels away or wraps layers of the cocoon depending on the changing need to manipulate engagement. The perceived reality of a virtual environment is varied by making the virtual environment correspond more closely to the extant environmental conditions—exposing the crew to a virtual experience of the actual conditions in the situation at the moment—when engagement wanes, and less closely—buffering the crew from experiencing the environment—when engagement is already adequate. In this way, engagement is managed by managing immersion in the flight experience, producing varying degrees of affective appreciation of the situation by manipulating the compelling nature of the environment.

A second embodiment is a variation of the first embodiment that involves modulating the relative clarity of the background panorama versus foreground instruments (which stay at full clarity) with the panorama becoming more visible with declining alertness to counteract disengagement. Differential highlighting of areas of the display to enhance attention to stimuli of current operational significance are superimposed on the overall uniform degree of clarity of the panorama. This embodiment provides a means of maintaining alertness within an optimal range, while at the same time providing additional flight-related information with which to be engaged.

A third embodiment of the innovation is an adaptive virtual ambience technology that comprises a system of wearable components and/or wearable devices 110 that monitor neural and cardiac signals, computes indices of cognitive and sympathetic arousal and transmits those indices to a subsystem, such as the local computing system 150, in an individual's sleep quarters. The subsystem may cause user interface devices 160, the augmented reality devices 170 or a combination of the user interface devices 160 and the augmented reality devices including an augmented reality panorama to display sleep-inducing images. The images may become dimmer and/or take on more relaxing characteristics as the arousal indices indicate less arousal, as determined by the biocybernetic adaptation and biofeedback training engine 154.

A fourth embodiment of the innovation is an adaptive virtual ambience technological system that comprises a system of wearable components and/or wearable devices 110 that monitor neural and cardiac signals, computes indices of arousal and engagement and transmits those estimates to a subsystem. For example, components of the local computing system 150 may be integrated into an autonomous vehicle deck environment. The subsystem may cause a synthetic panorama of the surrounding landscape/skyscape/traffic/terrain to be displayed as a backdrop to vehicle deck instrumentation on unused interior panel real estate such as via the user interface devices 160, the augmented reality devices 170, and/or a combination of the user interface devices 160 and the augmented reality devices 170. Sections of the panorama may be added or removed by the biocybernetic adaptation and biofeedback training engine 154 as requirements for engagement fluctuate, driven by fluctuations in the measured driver engagement state combined with evolving changes in the necessity for engagement in the vehicle management task.

A fifth embodiment of the innovation is a driver momentary readiness assessment technology that comprises a system of wearable components and/or wearable devices 110 that monitor neural and cardiac signals, computes indices of arousal and engagement and transmits those estimates to a subsystem in an autonomous vehicle deck environment (e.g., the local computing system 150). The estimates indicate to the system whether or not the occupant is prepared to take control of the system at critical junctures, such as a deer or other object suddenly appearing in the driving path, and whether the system needs to get the occupant's attention. If the occupant is not prepared, the system executes an automatic “plan B” to “help bring the operator gradually into awareness.

A sixth embodiment of the innovation is a biofeedback technology that comprises a system of wearable components and/or wearable devices 110 that monitor physiological signals, computes indices of arousal and engagement and transmits those indices to a subsystem integrated into the local computing system 150, such as a video or computer game environment. This embodiment incorporates biofeedback into video or computer games in such a way that a player (e.g., the user 105) and/or other players are motivated to adopt and maintain healthy, productive, or emotionally sound physiological patterns and/or abandon those which are unhealthy, unproductive, or emotionally unsound. The game motivates the player to approach a desired physiological pattern and/or depart from an undesirable pattern by altering, both quantitatively and qualitatively, characteristics which are incidental to the game's objectives or difficulty, but rather change how rewarding the game-playing experience is.

The patterns on which the biofeedback is based can be quantitative, temporal or both. The games of the invention are video and computer games that, like ordinary video and computer games, have a particular set of goals or objectives. The biofeedback does not modify the game goals or difficulty level nor how easily these goals are attained. The game instead motivates the player to approach a desired physiological pattern and/or depart from an undesirable pattern by altering, both quantitatively and qualitatively, characteristics which are incidental to the game's objectives or difficulty, but rather change how rewarding the game-playing experience is.

To make such systematic reinforcement possible, two functional elements in the game software which are not ordinarily present in video or computer games, work together to arrive at modifications of the games' operation and appearance. For example, the biocybernetic adaptation and biofeedback training engine 154 may include one or more components, such as a pattern comparator 157, and a reward calculator 159. The pattern comparator 157 stores physiological pattern templates and continually calculates a value indicative of the proximity of the player's actual moment-to-moment physiological activity to desirable and/or undesirable patterns. The templates used for reference in such comparison may be generic norm patterns for the group that the player belongs to (for example, age and gender norms) or may be based on prior measurement of the player's own physiology while in a desired state. The pattern comparator 157 calculates a closeness score to one or more reference patterns, and sends this information to the reward calculator. The reward calculator 159 uses the scores of closeness to desirable or undesirable patterns to determine when, and which, rewards are added to or subtracted from the gaming experience (qualitative reward change) and in some cases how much of a particular reward is present (quantitative reward change).

Some examples of qualitative changes which might be implemented by the reward calculator 159 may include one or more of the following:
1. Novelty. Rather than continuing to encounter the same kind of game objects and tasks, new ones are introduced into the game.
2. Depth. The game graphics change from two-dimensional representation to three-dimensional to virtual reality depiction of the game environment and objects.
3. Vividness. The game environment gains more features and richness.
4. Speed. The pace of the game is faster, without changing the relative advantage for the player. Thus, even though the game's difficulty does not change, the game becomes more exciting.
5. Appearance and expressiveness of objects. Objects, such as other persons, may become more expressive or colorful. Thus, characters in the game who were previously silent may begin to talk, or may show more facial expressions or body movement indicative of emotion, or characters acting in a straight-laced fashion may exhibit amusing behavior.
6. Music. The game may go from being silent to simple melody to have a rich and arousing score accompanying the action.
7. Impact. The player's actions may have an increasing degree of visual impact on the game objects and environment. In the case of a space-battle, the impact of shooting an alien flying saucer (while not becoming easier or more difficult to do so) may be altered from it simply fading out, to it lighting up and then blinking out, to it disintegrating in a fantastic explosion.
8. Type of environment. The terrain and cityscapes may change to add interest and richness to the experience.
9. Flexibility in the player's game movement. The player may go from being restricted to predefined paths to being able to move around more along the path, which allows more exploration and is more interesting.
10. Perspective/View. The player may go from seeing the character or object representing herself or himself (for example, a character person or a vehicle controlled by the player) as a small part of the overall game picture, to being the center of the game around which everything else (objects and environment) flows, to being inside the same character or object (first person view), which is the most engaging and interesting.
11. Dimensionality. A player may go from being limited to one dimension of the game to being able to go between dimensions, each dimension having different objects, tasks, and environment.
12. Status and nature of the player's character. The character representing the player may go from having a low status (for example, servant) to having a high status (for example, prince or princess). Although this may not alter the game difficulty or progress, higher status is more rewarding (this will be especially important in games of role-playing nature). Also, rewards may be increased by giving the player more and more ability to define and refine his or her own character, giving it a name, designing its looks, etc.
13. Reward choices. The player may go from having no overt choices over which rewards are added or subtracted, to being able to add or retain the one he or she likes best from a menu of game characteristics to be added or subtracted. This may be a “meta-reward”, for example, granted for showing good maintenance of a healthy physiological pattern over an extended time period during a play session.
14. Superpowers. The ordinary powers of the player's character or vehicle in a video game may be turbocharged, enhanced, or boosted from normal to supernatural or magical. The player's character may be given extra powers beyond those which are ordinarily available, that is, supernormal sensory, perceptual, cognitive, motor abilities, without giving the player a performance advantage in the game. For example, the player may be rewarded with X-ray vision, making for a richer visual experience. The player's vehicle may be given extra powers beyond those which are ordinarily available without giving the player a performance advantage in the game. For example, the player's magic carpet may fly higher, giving a more exciting view. Enhancing capability may involve matching the super functions of the player's character to recognized experiential characteristics of brainwave states. For example, increases in brainwave activity associated with daydreaming may enable richer visualization or vision or x-ray vision, increases in brainwave activity associated with mental effort may enable more capable artificial intelligence in the character, and increases in brainwave activity associated with stillness or graceful movement may enable smoother character motion. Any performance advantage that would be realized as a result of the enhanced capabilities would be offset by new challenges to match the enhanced capability, resulting in little change in the overall difficulty of the game for the player.

Based on the calculation by the physiological pattern comparator 157, a symbol or other representation is continually present and updated on the game's display screen, informing the player from moment to moment how distant or close he or she is from the threshold for the next reward modification (whether addition or subtraction). Between periods where new qualitative reward is added and subtracted (based upon a certain increment of quantitative change in the closeness between the player's current physiological pattern and the reference patterns in use by the physiological pattern comparator), the graphic representation of the closeness may correspond to quantitative changes in the same qualitative category. For example, after a player has earned addition of music accompaniment to the game, the music may become steadily richer and more satisfying until the threshold for the next qualitative reward addition is earned. If the player has also earned choices between reward categories, a graphic indicating what game characteristics the player can currently choose between is also presented on the display screen. At any given reward stage, one player may prefer to switch to a first-person view, for example, whereas another player might prefer sticking to third-person view but selecting to have music on instead. In this way, customizability of rewards to individual preferences becomes in and of itself another type of reward.

As discussed above, the system 100 may include at least two interacting sets of components. For example, the wearable devices 110 may include wearable devices 110 and/or environment-embedded components (e.g., the biocybernetic adaptation and biofeedback training engine 154) that track a traveler's physiological signals, compute estimates of their cognitive and emotional states and communicate the estimates to components embedded in the environments/settings through which the traveler moves (local travel within a domicile or multi-room facility or distant travel from one location to another geospatial location). Further, the system may include components embedded in the environments through which the traveler moves, that modify aspects of the environments in such a way as to encourage/reinforce neurophysiological self-regulation.

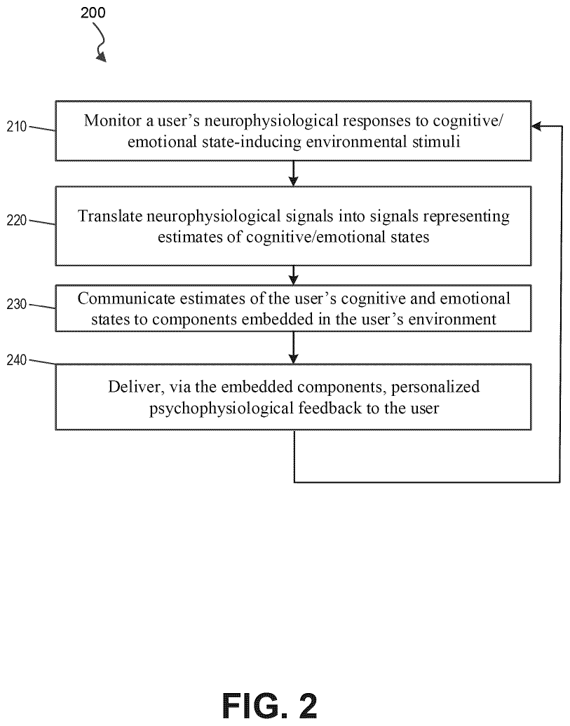

FIG. 2 shows an illustrative biocybernetic adaptation and biofeedback training method 200. At 210, via the wearable devices 110 and/or one or more environmental devices communicatively coupled to the wearable devices 110 may create a historical record of a user's neurophysiological responses to cognitive/emotional state-inducing environmental stimuli (e.g., probes). In some cases, the environmental stimuli may be a product of the environment local to the user. Alternatively, or additionally, the environmental stimuli may be generated by a biocybernetic adaptation and biofeedback training engine. At 220, the biocybernetic adaptation and biofeedback training engine 154 may translate neurophysiological signals into signals representing estimates of cognitive/emotional states by comparing with historical record of the user's neurophysiological responses to environmental probes. In some cases, the biocybernetic adaptation and biofeedback training engine 154 may be integrated into a local computing system 150, one or more wearable devices 110 (e.g., the mobile phone 116), or a combination of devices. At 230, estimates of the user's cognitive and emotional states may be communicated to components embedded in the environments through which the traveler moves.

At 240, the user interface devices 160 and/or the augmented reality devices 170 may deliver psychophysiological feedback to the user 105, that may be personalized based upon the user's record of responses, such as by modifying, in response to the user's real-time responses, components of an environment that are unique to the local environment. For example, augmented reality devices 170 may be integrated into an immediate environment around the user 105, such as within or upon surfaces of an airplane cockpit, an automobile interior, walls and items within a user's sleeping quarters, and/or the like. Modifying components present within each environment is enabled by incorporating within the components, a mechanism that may be termed biocybernetic adaptation or modulation—a mechanism that is not represented pervasively across environments in any prior invention.

The biocybernetic adaptation and/or modulation system may be pervasive and decentralized such that any physiological adaptation is not happening only in one specific context but instead, specific adaptations are created based on a set of finite and previously defined contexts. Adaptive ambient feedback may allow for continuous and subtle feedback to a user instead of creating limited and slightly noticeable feedback to persuade users. The system may modify the user's surrounding ambience in their environment such as by modulating visual, olfactory, haptic, and auditory elements and encouraging the user to improve specific present and on the fly self-regulation skills. Long-term learning for the user may be enabled by the biocybernetic adaptation and biofeedback training system using intelligent algorithms. For example, the system may integrate algorithms capable of learning about users' physiological responses in different moments and contexts to create more refined and robust adaptation each time a same or similar set of conditions exist.

A novel aspect of the biocybernetic adaptation and biofeedback training system may involve deployment of present technologies to regulate a user's state in a current situation and to train self-regulation skill over time by providing cognitive/emotional state feedback via changes in the functioning of these technological components in the environments through which the user transits. As such, the biocybernetic adaptation and biofeedback training system may be applicable to multiple sectors and applications. For example, the biocybernetic adaptation and biofeedback training system may enhance interaction and system intelligence when using virtual and augmented reality. For example, the use of physiologically adaptive systems that are ubiquitous and integration of wearable devices enhances the interaction when using technologies for extended realities (e.g., virtual or augmented reality). By empowering the system with capabilities to collect, interpret, and create real-time adaptations based on detected conscious or unconscious human states, it can foster a more robust interaction paradigm and enhance the information flow and technology usability. As such, technology intensive companies may integrate such features in social media networks, online or localized entertainment and/or game play, and integration into multiple internal and external human interface technologies.

Additionally, the biocybernetic adaptation and biofeedback training system may be used for reducing stress and improving automatization in autonomous vehicles. For example, the system may be used to detect or otherwise identify specific human states associated with psychological states of drivers when using autonomous vehicles. As such, these systems may reduce the workload and/or improve the learning curve when using autonomous cars. When the autonomous vehicle detects possible threatening levels of a human state (e.g., stress, workload), the biocybernetic adaptation and biofeedback training system can create adaptations in the surrounding displays to facilitate the transition to a more relaxing and calm state that enhances the driving experience. While automobiles are discussed, other autonomous vehicles may similarly benefit from use of the biocybernetic adaptation and biofeedback training system.

Additionally, the biocybernetic adaptation and biofeedback training system may improve game user experience by adding biofeedback technologies to games and interactive applications. Games and game play have been innovated with interfaces that connect different intentions of players with the virtual environments, characters, and narratives created. Biofeedback technologies have been previously used to create gaming experiences that can connect the human body and mind in a more natural and meaningful way. The biocybernetic adaptation and biofeedback training system and methods may be integrated in games and interactive applications to create experiential interactions that provide stronger, more emotionally connected, and interactive experiences than is currently possible with conventional input systems (e.g., handheld controllers).

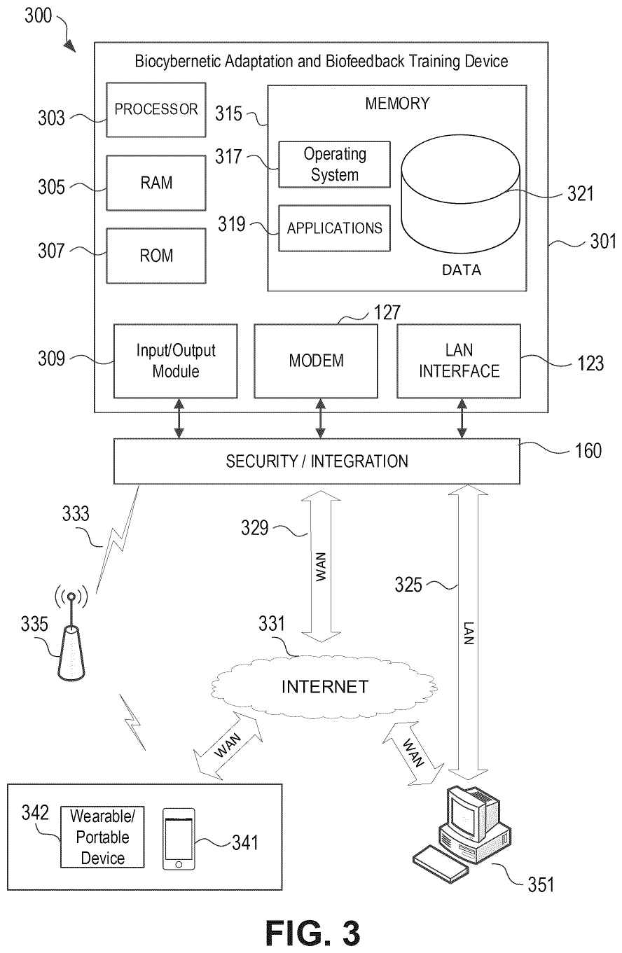

FIG. 3 shows an illustrative operating environment 300 in which various aspects of the present disclosure may be implemented in accordance with one or more example embodiments. Referring to FIG. 3, a computing system environment 300 may be used according to one or more illustrative embodiments. The computing system environment 300 is only one example of a suitable computing environment and is not intended to suggest any limitation as to the scope of use or functionality contained in the disclosure. The computing system environment 300 should not be interpreted as having any dependency or requirement relating to any one or combination of components shown in the illustrative computing system environment 300.

The computing system environment 300 may include an illustrative biocybernetic adaptation and biofeedback training device 301 having a processor 303 for controlling overall operation of the biocybernetic adaptation and biofeedback training device 301 and its associated components, including a Random-Access Memory (RAM) 305, a Read-Only Memory (ROM) 307, a communications module 309, and a memory 315. The biocybernetic adaptation and biofeedback training device 301 may include a variety of computer readable media. Computer readable media may be any available media that may be accessed by the biocybernetic adaptation and biofeedback training device 301, may be non-transitory, and may include volatile and nonvolatile, removable, and non-removable media implemented in any method or technology for storage of information such as computer-readable instructions, object code, data structures, program modules, or other data. Examples of computer readable media may include Random Access Memory (RAM), Read Only Memory (ROM), Electronically Erasable Programmable Read-Only Memory (EEPROM), flash memory or other memory technology, Compact Disk Read-Only Memory (CD-ROM), Digital Versatile Disk (DVD) or other optical disk storage, magnetic cassettes, magnetic tape, magnetic disk storage or other magnetic storage devices, or any other medium that can be used to store the desired information and that can be accessed by the biocybernetic adaptation and biofeedback training device 301.

Although not required, various aspects described herein may be embodied as a method, a computing (e.g., data transfer) system, or as a computer-readable medium storing computer-executable instructions. For example, a computer-readable medium storing instructions to cause a processor to perform steps of a method in accordance with aspects of the disclosed embodiments is contemplated. For example, aspects of method steps disclosed herein may be executed by the processor 303 of the biocybernetic adaptation and biofeedback training device 301. Such a processor may execute computer-executable instructions stored on a computer-readable medium.

Software may be stored within the memory 315 and/or other digital storage to provide instructions to the processor 303 for enabling the biocybernetic adaptation and biofeedback training device 301 to perform various functions as discussed herein. For example, the memory 315 may store software used by the biocybernetic adaptation and biofeedback training device 301, such as an operating system 317, one or more application programs 319 (e.g., a web browser application), and/or an associated database 321. In addition, some, or all of the computer executable instructions for the biocybernetic adaptation and biofeedback training device 301 may be embodied in hardware or firmware. Although not shown, the RAM 305 may include one or more applications representing the application data stored in the RAM 305 while the biocybernetic adaptation and biofeedback training device 301 is on and corresponding software applications (e.g., software tasks) are running on the biocybernetic adaptation and biofeedback training device 301.

The communications module 309 may include a microphone, a keypad, a touch screen, and/or a stylus through which a user of the biocybernetic adaptation and biofeedback training device 301 may provide input, and may include one or more of a speaker for providing audio output and a video display device for providing textual, audiovisual, and/or graphical output. The computing system environment 300 may also include optical scanners (not shown).

The biocybernetic adaptation and biofeedback training device 301 may operate in a networked environment supporting connections to one or more remote computing devices, such as the computing devices 341, 342, and 351. The computing devices 341, 342, and 351 may be wearable computing devices, personal computing devices or servers that include any or all of the elements described above relative to the biocybernetic adaptation and biofeedback training device 301.

The network connections depicted in FIG. 3 may include a Local Area Network (LAN) 325 and/or a Wide Area Network (WAN) 329, as well as other networks. When used in a LAN networking environment, the biocybernetic adaptation and biofeedback training device 301 may be connected to the LAN 325 through a network interface or adapter in the communications module 309. When used in a WAN networking environment, the biocybernetic adaptation and biofeedback training device 301 may include a modem in the communications module 309 or other means for establishing communications over the WAN 329, such as a network 331 (e.g., public network, private network, Internet, intranet, and the like). The network connections shown are illustrative and other means of establishing a communications link between the computing devices may be used. Various well-known protocols such as Transmission Control Protocol/Internet Protocol (TCP/IP), Ethernet, File Transfer Protocol (FTP), Hypertext Transfer Protocol (HTTP) and the like may be used, and the system can be operated in a client-server configuration to permit a user to retrieve web pages from a web-based server. Any of various conventional web browsers can be used to display and manipulate data on web pages.

The disclosure is operational with numerous other computing system environments or configurations. Examples of computing systems, environments, and/or configurations that may be suitable for use with the disclosed embodiments include, but are not limited to, personal computers (PCs), server computers, hand-held or laptop devices, smart phones, multiprocessor systems, microprocessor-based systems, set top boxes, programmable consumer electronics, network PCs, minicomputers, mainframe computers, distributed computing environments that include any of the above systems or devices, and the like that are configured to perform the functions described herein.

What is claimed is:

1. A biocybernetic adaptation and biofeedback training system comprising:
a plurality of electronic devices associated with a user, wherein each electronic device of the plurality of electronic devices generate a signal associated with a physiological response of the user;
a computing device comprising:
at least one processor;
memory storing instructions that, when executed by the at least one processor, cause the computing device to:
receive, from at least one electronic device of the plurality of electronic devices, a first signal associated with a first physiological response of the user;
determine, based on the first signal, an estimate of a state of the user; and
cause, based on the estimate of the state of the user, presentation of an electronic representation of a psychophysiological stimuli via a second electronic device embedded in an environment local to the user.

2. The system of claim 1, wherein the state of the user comprises one or both of a cognitive state and an emotional state.

3. The system of claim 1, wherein the instructions further cause the computing device to send, to the second electronic device embedded in the environment local to the user, a signal associated with the estimate of the state of the user.

4. The system of claim 1, wherein the plurality of electronic devices associated with the user comprises a smart phone.

5. The system of claim 1, wherein the plurality of electronic devices associated with the user comprises one or more physiological sensor devices.

6. The system of claim 5, wherein at least one physiological sensor device generates a signal associated with a physiological state experienced by the user.

7. The system of claim 1, wherein the environment local to the user comprises a vehicle interior.

8. The system of claim 1, wherein the environment local to the user comprises a portion of an electronic gaming system.

9. The system of claim 1, wherein the second electronic device embedded in the environment local to the user comprises a virtual reality display.

10. The system of claim 1, wherein the instructions further cause the computing device to cause the virtual reality display to display a visual representation of a locality external to the environment local to the user.

11. The system of claim 10, wherein the environment local to the user comprises an interior space of a vehicle and the locality external to the environment local to the user comprises a space external to the vehicle.

12. A method comprising:
receiving, from at least one electronic device of a plurality of electronic devices associated with a user, a first signal associated with a first physiological response of the user;
determining, based on the first signal, an estimate of a state of the user comprising one or both of a cognitive state of the user and an emotional state of the user; and
causing, based on the estimate of the state of the user, presentation of an electronic representation of a psychophysiological stimuli via a second electronic device embedded in an environment local to the user.

13. The method of claim 12, further comprising sending, to the second electronic device embedded in the environment local to the user, a signal associated with the estimate of the state of the user.

14. The method of claim 12, wherein the plurality of electronic devices associated with the user comprises a smart phone.

15. The method of claim 13, wherein the plurality of electronic devices associated with the user comprises at least one physiological sensor device.

16. The method of claim 15, wherein the at least one physiological sensor device generates a signal associated with a physiological state experienced by the user.

17. The method of claim 12, wherein the second electronic device embedded in an environment local to the user comprises a virtual reality device associated with a gaming system.

18. The method of claim 17, wherein the virtual reality device generates one or more of visual feedback, haptic feedback, and audio feedback.

19. A computing device comprising:
at least one processor;
memory storing instructions that, when executed by the at least one processor, cause the computing device to:
receive, from at least one electronic device of a plurality of electronic devices, a first signal associated with a first physiological response of a user, wherein the plurality of electronic devices comprises one or more of a physiological sensor, a smart watch, and a smart phone;
determine, based on the first signal, an estimate of a state of the user; and
cause, based on the estimate of the state of the user, presentation of an electronic representation of a psychophysiological stimuli via a second electronic device embedded in an environment local to the user.

20. The computing device claim 19, wherein the second electronic device embedded in an environment local to the user comprises a virtual reality device associated with a gaming system and the virtual reality device generates one or more of visual feedback, haptic feedback, and audio feedback.
